(19) United States
(12) Patent Application Publication
(10) Pub. No.: US 2022/0061802 A1
(43) Pub. Date: Mar. 3, 2022

(54) SYSTEMS AND METHODS FOR IDENTIFYING FEATURES SENSED BY A VASCULAR DEVICE

(71) Applicant: KONINKLIJKE PHILIPS N.V., EINDHOVEN (NL)

(72) Inventors: Nathan C. FRANCIS, COLORADO SPRINGS, CO (US); Ryan Michael SOTAK, COLORADO SPRINGS, CO (US); Wade Allen BOWE, COLORADO SPRINGS, CO (US); Christian HAASE, HAMBURG (DE)

(21) Appl. No.: 17/422,763

(22) PCT Filed: Jan. 2, 2020
(86) PCT No.: PCT/EP2020/050002

§ 371 Date: Jul. 14, 2021

Related U.S. Application Data
(60) Provisional application No. 62/792,787, filed on Jan. 15, 2019.

(51) Int. Cl. A61B 8/12; A61B 8/00; A61B 8/08

(52) U.S. Cl. CPC A61B 8/12; A61B 8/085; A61B 8/463

(57) ABSTRACT

Embodiments disclosed herein relate to systems and methods for identifying features sensed by a vascular device. In an embodiment, a vascular device configured to identify features sensed by the vascular device comprises and imaging device and a processing device. The imaging device is configured to be disposed in a vascular space and send at least one signal corresponding to an image of the vascular space. The processing device is electronically coupled to the imaging device and is configured to: receive the at least one signal corresponding to the image of the vascular space; determine at least one feature included in the image; identify at least a portion of the feature using a graphical representation; and output the graphical representation identifying the portion of the feature to a display device.

### FIELD OF THE DISCLOSURE

The systems and devices described herein generally relate to vascular treatment systems and devices including intravascular imaging capabilities, and more specifically relate to cardiac lead extraction systems and devices including intravascular imaging capabilities.

### BACKGROUND

Surgically implanted cardiac implantable electronic devices (CIEDs), such as pacemakers and defibrillators, play an important role in the treatment of heart disease. In the 50 years since the first pacemaker was implanted, technology has improved dramatically, and these systems have saved or improved the quality of countless lives. Pacemakers treat slow heart rhythms by increasing the heart rate or by coordinating the heart's contraction for some heart failure patients. Implantable cardioverter-defibrillators stop dangerous rapid heart rhythms by delivering an electric shock.

Some CIEDs typically include a timing device and a lead, which are placed inside the body of a patient. One part of the system is the pulse generator containing electric circuits and a battery, usually placed under the skin on the chest wall beneath the collarbone. To replace the battery, the pulse generator must be changed by a simple surgical procedure every 5 to 10 years. Another part of the system includes the wires, or leads, which run between the pulse generator and the heart. In a pacemaker, these leads allow the device to increase the heart rate by delivering small timed bursts of electric energy to make the heart beat faster. In a defibrillator, the lead has special coils to allow the device to deliver a high-energy shock and convert potentially dangerous rapid rhythms (ventricular tachycardia or fibrillation) back to a normal rhythm. Additionally, the leads may transmit information about the heart's electrical activity to the pacemaker.

For both functions, leads must be in contact with heart tissue. Most leads pass through a vein under the collarbone that connects to the right side of the heart (right atrium and right ventricle). In some cases, a lead is inserted through a vein and guided into a heart chamber where it is attached with the heart. In other instances, a lead is attached to the outside of the heart. To remain attached to the heart muscle, most leads have a fixation mechanism, such as a small screw and/or hooks at the end.

Within a relatively short time after a lead is implanted into the body, the body's natural healing process forms scar tissue along the lead and possibly at its tip, thereby fastening it even more securely in the patient's body. Leads usually last longer than device batteries, so leads are simply reconnected to each new pulse generator (battery) at the time of replacement. Although leads are designed to be implanted permanently in the body, occasionally these leads must be removed, or extracted. Leads may be removed from patients for numerous reasons, including but not limited to, infections, lead age, and lead malfunction.

Removal or extraction of the lead may be difficult. As mentioned above, the body's natural healing process forms scar tissue over and along the lead, and possibly at its tip, thereby encasing at least a portion of the lead and fastening it even more securely in the patient's body. In addition, the lead and/or tissue may become attached to the vasculature wall. Both results may, therefore, increase the difficulty of removing the leads from the patient's vasculature.

A variety of tools have been developed to make lead extraction safer and more successful. Current lead extraction techniques include mechanical traction, mechanical devices, and laser devices. Mechanical traction may be accomplished by inserting a locking stylet into the hollow portion of the lead and then pulling the lead to remove it. An example of such a lead locking device is described and illustrated in U.S. Pat. No. 6,167,315 to Coe et al., which is hereby incorporated herein by reference in its entirety for all that it teaches and for all purposes.

A mechanical device to extract leads may include one or more flexible tubes called sheaths that pass over the lead and/or the surrounding tissue. One of the sheaths may include a tip having a dilator, a separator and/or a cutting blade, such that upon advancement, the tip (and possibly the sheath cooperate to) dilates, separates and/or cuts to separate the scar tissue from other scar tissue including the scar tissue surrounding the lead. In some cases, the tip (and sheath) may also separate the tissue itself from the lead. Once the lead is separated from the surrounding tissue and/or the surrounding tissue is separated from the remaining scar tissue, the lead may be inserted into a hollow lumen of the sheath for removal and/or be removed from the patient's vasculature using some other mechanical devices, such as the mechanical traction device previously described in United States Patent Publication No. 2008/0154293 to Taylor, which is hereby incorporated herein by reference in its entirety for all that it teaches and for all purposes.

Some lead extraction devices include mechanical sheaths that have trigger mechanisms for extending the blade from the distal end of the sheath. An example of such devices and method used to extract leads is described and illustrated in U.S. Pat. No. 5,651,781 to Grace, which is hereby incorporated herein by reference in its entirety for all that it teaches and for all purposes. Another example of these device that has a trigger mechanism for extending the blade from the distal end of the sheath is described and illustrated in United States Patent Publication No. 2014/0277037 having application Ser. No. 13/834,405 filed Mar. 14, 2013, which is hereby incorporated herein by reference in its entirety for all that it teaches and for all purposes.

Lead extraction procedures typically include the use of fluoroscopy to facilitate visualization and tracking of lead extraction devices within a patient's body. However, fluoroscopy has several disadvantages. For example, fluoroscopy provides poor contrast for soft tissues. As another example, fluoroscopy provides two-dimensional imaging of three-dimensional anatomy. These disadvantages inhibit physicians from understanding the anatomy of a specific patient's body. In other cases, lead extraction procedures include the use of an imaging catheter in addition to lead extraction devices. However, such imaging catheters typically require another venous access point and a second operator, and the second operator must attempt to spatially register the lead extraction device to the imaging catheter. Furthermore, imaging catheters are typically poorly suited for lead extraction procedures in terms of, for example, form factor, visual field, and/or accessibility.

Accordingly, it is desirable to provide improved vascular treatment systems and devices including intravascular imaging capabilities.

### SUMMARY

The present disclosure presents a vascular treatment system that includes an imaging device. An imaging device is configured to be disposed in the treatment space and send a signal corresponding to an image of the treatment space. A display is in operative communication with the imaging device and is configured to provide the image of the treatment space to a system user. Example embodiments include but are not limited to the following:

A vascular device configured to identify features sensed by the vascular device, comprising: an imaging device configured to be disposed in a vascular space and send at least one signal corresponding to an image of the vascular space; and a processing device electronically coupled to the imaging device, the processing device configured to: receive the at least one signal corresponding to the image of the vascular space; determine at least one feature included in the image; identify at least a portion of the feature using a graphical representation; and output the graphical representation identifying the portion of the feature to a display device.

The vascular device according to the previous paragraph, wherein the imaging device is an ultrasound device, and further comprising an imaging device coupled to the ultrasound device.

The vascular device according to any of the previous paragraphs, wherein to identify the portion of the feature with the graphical representation, the processing device identifies the portion of the feature with an identifiable line.

The vascular device according to any of the previous paragraphs, wherein the identifiable line is a colored line.

The vascular device according to any of the previous paragraphs, wherein the feature is at least partially surrounded by the identifiable line.

The vascular device according to any of the previous paragraphs, wherein the feature is at least partially overlaid by the identifiable line.

The vascular device according to any of the previous paragraphs, wherein to determine the at least one feature, the processing device determines at least one of: a vessel wall boundary, a lead, fibrotic adhesions to cardiovascular segments, calcium in fibrotic adhesions, thrombus within cardiovascular segments, vegetation within cardiovascular segments, and boundaries between a vessel wall and at least one of pericardium and pleura.

The vascular device according to any of the previous paragraphs, wherein the processing device is further configured to: overlay the image with a plurality of dots; determine at least one distance between two dots of the plurality of dots; and calculate dimensions of the at least one feature using the determined distance between the two dots.

The vascular device according to any of the previous paragraphs, wherein the processing device is further configured to: overlay the image with a plurality of dots; determine at least one distance between two dots of the plurality of dots; and calculate a distance between two features of the at least one feature using the determined distance between the two dots.

The vascular device according to any of the previous paragraphs, wherein to determine the at least one feature included in the image, the processing device uses machine learning.

The vascular device according to any of the previous paragraphs, wherein to determine the at least one feature included in the image, the processing device access a lookup table.

The vascular device according to any of the previous paragraphs, wherein the processing device is further configured to output a notification when at least one event occurs.

The vascular device according to the previous paragraph, wherein to determine the at least one feature, the processing device determines a lead and a vessel wall, and one of the at least one event occurs when the lead extends outside the vessel wall.

A method for identifying features sensed by the vascular device, the method comprising: receiving at least one signal corresponding to an image sensed by a vascular device; determining at least one feature included in the image; identifying at least a portion of the feature with a graphical representation; and outputting the graphical representation identifying the portion of the feature to a display device.

The method according to the previous paragraph, wherein identifying a portion of the feature with the graphical representation comprises identifying the portion of the feature with an identifiable line.

The method according to any of the previous paragraphs, wherein identifying the portion of the feature with an identifiable line comprises surrounding the feature with the identifiable line.

The method according to any of the previous paragraphs, wherein identifying the portion of the feature with an identifiable line comprises overlaying the feature with the identifiable line.

The method according to any of the previous paragraphs, further comprising: overlaying the image with a plurality of dots; determining at least one distance between two dots of the plurality of dots; and calculating dimensions of the at least one feature using the determined distance between the two dots.

The method according to any of the previous paragraphs, further comprising: overlaying the image with a plurality of dots; determining at least one distance between two dots of the plurality of dots; and calculating a distance between two features included in the image using the determined distance between the two dots.

The method according to any of the previous paragraphs, further comprising outputting a notification when at least one event occurs.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (for example, X₁ and X₂) as well as a combination of elements selected from two or more classes (for example, Y₁ and Zₒ).

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

The term “means” as used herein shall be given its broadest possible interpretation in accordance with 35 U.S.C. Section 112(f). Accordingly, a claim incorporating the term “means” shall cover all structures, materials, or acts set forth herein, and all the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

This patent file contains at least one drawing executed in color. Copies of this patent with color drawing(s) will be provided by the Office upon request and payment of the necessary fee.

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a schematic illustration of a vascular treatment system according to an embodiment of the present disclosure.

FIG. 2 is a side view of an exemplary vascular treatment device of vascular treatment systems according to embodiments of the present disclosure.

FIG. 3A is a partial side view of a distal end portion of an exemplary vascular treatment device according to embodiments of the present disclosure.

FIG. 3B is an end view of the distal end portion of the vascular treatment device of FIG. 3A.

FIG. 4A is a partial side view of a distal end portion of another exemplary vascular treatment device according to embodiments of the present disclosure.

FIG. 4B is an end view of the distal end portion of the vascular treatment device of FIG. 4A.

FIG. 5A is a partial side view of a distal end portion of another exemplary vascular treatment device according to embodiments of the present disclosure.

FIG. 5B is an end view of the distal end portion of the vascular treatment device of FIG. 5A.

FIG. 6A is a partial side view of a distal end portion of another exemplary vascular treatment device according to embodiments of the present disclosure.

FIG. 6B is an end view of the distal end portion of the vascular treatment device of FIG. 6A.

FIG. 7A is a partial side view of a distal end portion of another exemplary vascular treatment device according to embodiments of the present disclosure.

FIG. 7B is an end view of the distal end portion of the vascular treatment device of FIG. 7A.

FIG. 8 is a schematic illustration of an exemplary controller of the vascular treatment system of FIG. 1.

FIG. 9 is a first illustration of an exemplary image of a vascular space generated by the controller of FIG. 8, featuring a vessel wall boundary, a lead, and an adhesion in the vascular space.

FIG. 10 is a second illustration of the exemplary image of the vascular space generated by the controller of FIG. 8, featuring the lead and the adhesion.

FIG. 11 is a third illustration of the exemplary image of the vascular space generated by the controller of FIG. 8, featuring the lead and the vessel wall boundaries.

FIG. 12 is a fourth illustration of the exemplary image of the vascular space generated by the controller of FIG. 8, featuring an atrial wall and a pericardium.

FIGS. 13A-13C illustrate exemplary notifications generated by the controller of FIG. 8 using colored indicators.

FIG. 14 is a flow chart of an exemplary method of identifying anatomical features sensed by the controller of FIG. 8.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

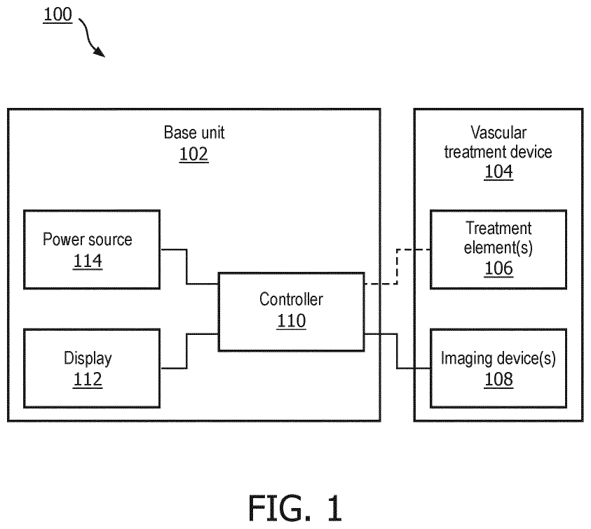

The present disclosure relates generally to vascular treatment systems and devices including intravascular imaging capabilities. FIG. 1 illustrates a vascular treatment system 100 according to an embodiment of the present disclosure. The vascular treatment system 100 generally includes a base unit 102 this is configured to be disposed externally from a treatment space (for example, the vasculature of subject, such as a patient) and a vascular treatment device 104 that is configured to be at least partially disposed within the treatment space and provide treatment to the subject during a vascular surgical procedure. The vascular treatment device 104 may detachably couple to the base unit 102. Similarly, the vascular treatment device 104 may be a “single use” device, and the base unit 102 may be a “multiple use” unit. The vascular treatment device 104 includes one or more treatment elements 106 that interact with and modify vascular structures (for example, tissue, plaque deposits, and the like). The treatment elements 106 may be, for example, configured to physically engage and thereby modify vascular structures (more specifically, the treatment elements 106 may be cutting elements, shearing elements, dilating elements, or the like). As another example, the treatment elements 106 may be configured to emit energy that modifies vascular structures (more specifically, the treatment elements 106 may emit electrical energy or radiofrequency energy, or the treatment elements 106 may be optical fibers that emit laser energy).

The vascular treatment device 104 further includes one or more imaging devices 108 that facilitate providing images of the treatment space to a system user (for example, a physician). The imaging devices 108 may be, for example, ultrasound imaging devices (as more specific examples, piezo-ceramic devices, piezo-film devices, piezoelectric micromachined ultrasonic transducer (PMUT) devices, or capacitive micromachined ultrasonic transducer (CMUT) devices), visible light imaging devices, infrared light imaging devices, spectroscopy imaging devices, impedance mapping imaging devices, or the like. Generally, the imaging devices 108 facilitate providing images of the treatment space to the system user. For example, the imaging devices 108 may send signals from which images of the treatment space may be generated. In some embodiments, the imaging devices 108 may be used in a phased-array manner. In some embodiments, the imaging devices 108 may include a coating to inhibit abrasion of the imaging devices 108 during advancement within a subject. For embodiments in which the imaging devices 108 are optical devices, the coating may be relatively hard and optically clear. For embodiments in which the imaging devices 108 are acoustic devices, the coating may be an acoustic matching layer to the external environment. As specific examples, the coatings may include silicon-based epoxies, polymer-based materials, or the like.

With continued reference to FIG. 1, the base unit 102 includes a controller 110 that is in operative communication with the imaging devices 108 and/or the treatment elements 106 (for example, via wired or wireless communication). The controller 110 is also in operative communication with a display 112 (for example, an LCD display, an LED display, or the like) that provides images of the treatment space. The controller 110 is also in operative communication with a power source 114 (for example, a cord for coupling to the base unit 102 to an external outlet, one or more batteries, or the like), and the controller 110 may thereby deliver power to the imaging devices 108, the treatment elements 106, and/or the display 112. In embodiments in which the treatment elements 106 of vascular treatment devices are optical fibers that emit laser energy, the base unit 102 may also include components for generating laser energy. More specifically, the base unit 102 may be similar to the Spectranetics CVX-300® Excimer Laser System, which is available from the Koninklijke Philips N.V.

Vascular treatment systems according to embodiments of the present disclosure may take other forms. For example, in some embodiments vascular treatment devices may carry one or more of a controller, a display, or a power source. As another example, in some embodiments vascular treatment devices may include combinations of various types of treatment elements and/or imaging devices.

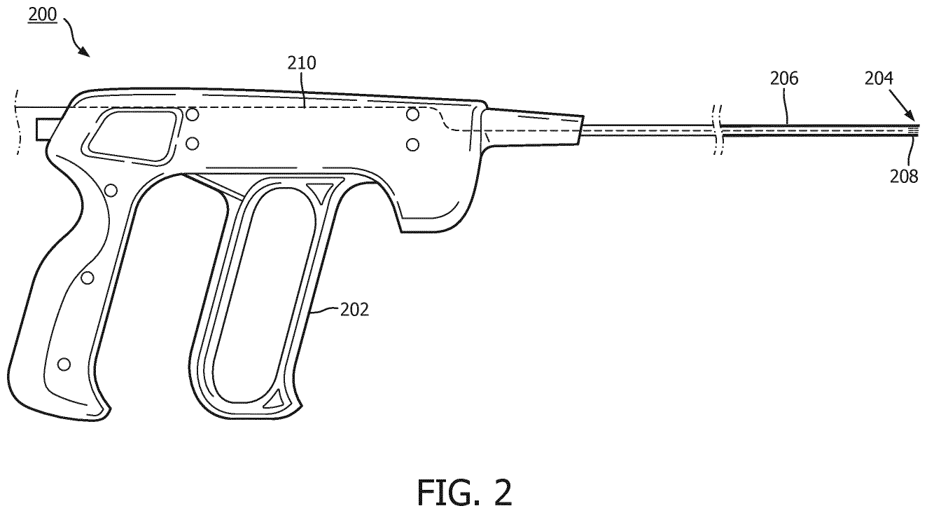

Vascular treatment devices forming part of systems according to embodiments of the present disclosure may take various forms. For example, and referring to FIG. 2, an exemplary embodiment of a vascular treatment device is illustrated. The vascular treatment device is a cardiac lead extraction device 200 and may be similar to any of the extraction devices disclosed in United States Patent Application Publication No. 2017/0172622 having application Ser. No. 15/442,006 filed Feb. 24, 2017 or United States Patent Application Publication No. 2015/0164530 having application Ser. No. 14/635,742 filed Mar. 2, 2015, which is hereby incorporated herein by reference in its entirety for all that it teaches and for all purposes. That is, the lead extraction device 200 includes a trigger 202 that is actuatable to drive a treatment element, specifically a rotatable cutting tip (not shown) disposed at a distal end portion 204 of a sheath assembly 206, and thereby separate tissue from an adjacent lead. In addition, the lead extraction device 200 includes one or more imaging devices 208 disposed at the distal end portion 204 of the sheath assembly 206. The lead extraction device 200 may further include one or more cables 210 for operatively coupling the device (more specifically, the imaging devices 208) to a base unit. Alternatively, the imaging devices 208 may be wirelessly operatively coupled to a base unit. In other embodiments, vascular treatment devices may facilitate removal or manipulation of other indwelling objects (for example, inferior vena cava filters).

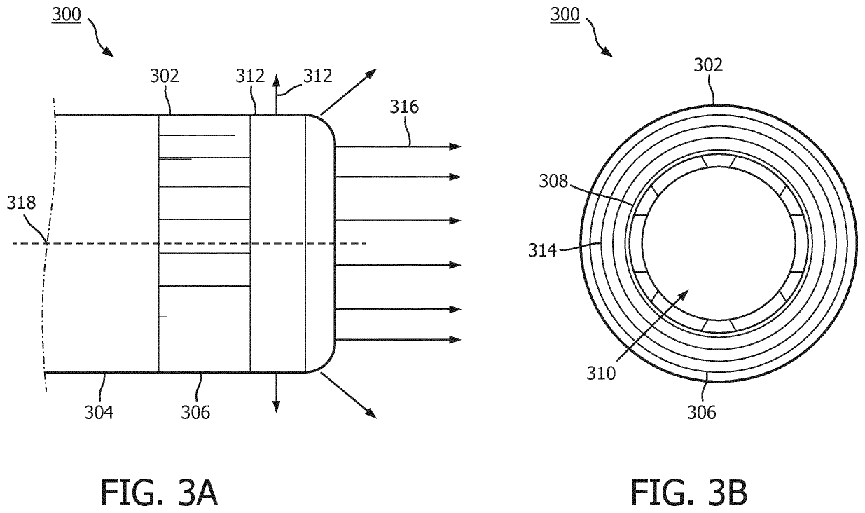

Arrangements of imaging devices and treatment elements of systems and devices according to embodiments of the present disclosure, including the arrangement at the distal end portion of the lead extraction device, may take various forms. For example, and referring to FIGS. 3A and 3B, an exemplary embodiment of a distal end portion 300 of a lead extraction device is illustrated. The distal end portion 300 is part of a sheath assembly 302 that includes an outer sheath 304 or jacket and an outer band or distal tip 306 coupled to and extending distally from the outer sheath 304. An inner sheath (not shown) is rotatably carried within the outer sheath 304, and a cutting tip 308 couples to and extends distally from the inner sheath. As such, the cutting tip 308 is rotatable relative to the outer band 306 to cut and separate tissue from an adjacent lead. The cutting tip 308 may also selectively extend distally relative to the outer band 306 to cut and separate tissue from the lead. The cutting tip 308 and the inner sheath also define an inner lumen 310 for receiving such a lead.

The distal end portion 300 of the lead extraction device further includes a first imaging device 312 (see FIG. 3A) and a second imaging device 314 (see FIG. 3B), which may specifically be any of the imaging devices described herein. Generally, the first imaging device 312 and the second imaging device 314 send signals corresponding to an image of the treatment space, and a display in operative communication with the imaging devices (shown elsewhere) provides the image of the treatment space to a user. The first imaging device 312 is carried by the outer band 306. The first imaging device 312 may have a generally annular shape. The first imaging device 312 may be disposed within the outer band 306 and radially and concentrically outward of the cutting tip 308. The first imaging device 312 may be disposed to provide the image of the treatment space with a first viewing centerline 320 that is substantially perpendicular to a longitudinal axis 318 of the sheath assembly 302 (that is, perpendicular ±5 degrees). Stated another way, the first imaging device 312 may be a transversely-viewing imaging device. The first imaging device 312 may provide a viewing cone of ±45 degrees from the centerline 316. The second imaging device 314 is carried by the outer band 306 distally relative to the first imaging device 312. The second imaging device 314 may have a generally annular shape. The second imaging device 314 may be disposed within the outer band 306 and radially and concentrically outward of the cutting tip 308. The second imaging device 314 may be disposed to provide the image of the treatment space with a second viewing centerline 316 that is substantially parallel to the longitudinal axis 318 (that is, parallel ±5 degrees). Stated another way, the second imaging device 314 may be a distally-viewing imaging device. The second imaging device 314 may provide a viewing cone of ±45 degrees from the centerline 320. In some embodiments, the first imaging device 312 and the second imaging device 314 may be recessed into the outer band 306 to inhibit abrasion of the imaging devices during advancement of the vascular treatment device within a subject. In some embodiments, the distal end portion 300 includes only one of the first imaging device 312 and the second imaging device 314. That is, in some embodiments distal end portions of vascular treatment devices according to the present disclosure include only a distally-viewing imaging device or only a transversely-viewing imaging device.

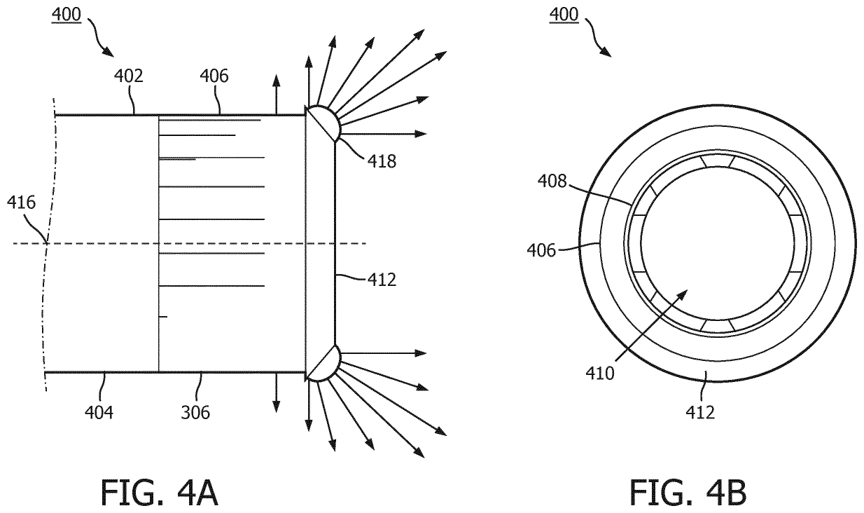

As another example and referring to FIGS. 4A and 4B, an exemplary embodiment of a distal end portion 400 of a lead extraction device is illustrated. The distal end portion 400 is part of a sheath assembly 402 that includes an outer sheath 404 or jacket and an outer band or distal tip 406 coupled to and extending distally from the outer sheath 404. An inner sheath (not shown) is rotatably carried within the outer sheath 404, and a cutting tip 408 couples to and extends distally from the inner sheath. As such, the cutting tip 408 is rotatable relative to the outer band 406 to cut and separate tissue from an adjacent lead. The cutting tip 408 may also selectively extend distally relative to the outer band 406 to cut and separate tissue from the lead. The cutting tip 408 and the inner sheath also define an inner lumen 410 for receiving such a lead.

The distal end portion 400 of the lead extraction device further includes an imaging device 412, which may specifically be any of the imaging devices described herein. Generally, the imaging device 412 send a signal corresponding to an image of the treatment space, and a display in operative communication with the imaging device 412 (shown elsewhere) provides the image of the treatment space to a user. The imaging device 412 is carried on an outer corner of the outer band 406. In some embodiments, the imaging device 412 is flush with the distal end of the outer band 406. More specifically, the imaging device 412 may be mounted to a chamfer (not shown) formed on the outer band 406. In some embodiments, the imaging device 412 is recessed relative to the outer band 406. The imaging device 412 may have a generally annular shape. The imaging device 412 may be disposed to provide the image of the treatment space with an acute viewing centerline 414 relative to a longitudinal axis 416 of the sheath assembly 402. The imaging device 412 may provide a viewing cone of ±45 degrees from the centerline 414. In some embodiments, the imaging device 412 is an ultrasound device, and the distal end portion 400 further includes an acoustic lens 418. Such an acoustic lens 418 facilitates “bending” ultrasound signals that are non-perpendicular to the imaging device 412 into a perpendicular direction relative to the imaging device 412. That is, the acoustic lens 418 facilitates simultaneously providing various viewing angles, such as a viewing angle that is substantially perpendicular to the longitudinal axis 416, a viewing angle along the centerline 414, and a viewing angle that is substantially parallel to the longitudinal axis 416.

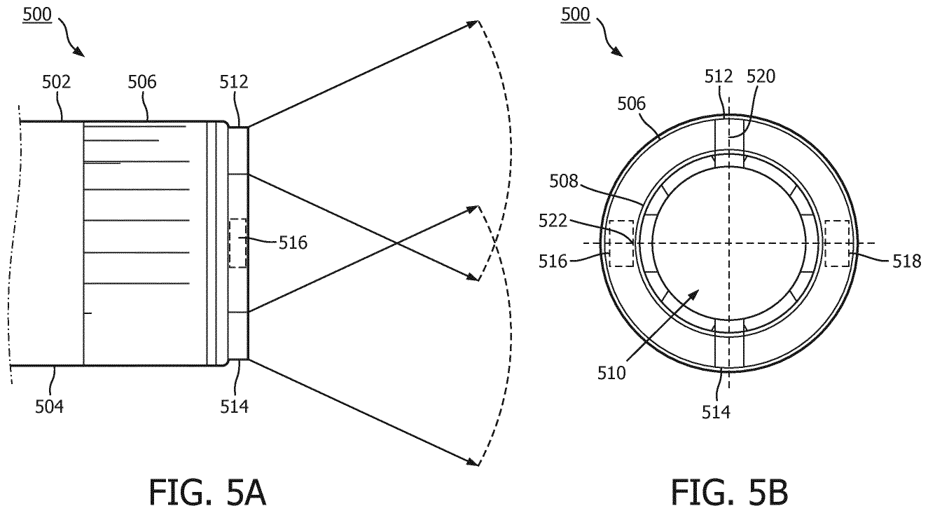

As another example and referring to FIGS. 5A and 5B, an exemplary embodiment of a distal end portion 500 of a lead extraction device is illustrated. The distal end portion 500 is part of a sheath assembly 502 that includes an outer sheath 504 or jacket and an outer band or distal tip 506 coupled to and extending distally from the outer sheath 504. An inner sheath (not shown) is rotatably carried within the outer sheath 504, and a cutting tip 508 couples to and extends distally from the inner sheath. As such, the cutting tip 508 is rotatable relative to the outer band 506 to cut and separate tissue from an adjacent lead. The cutting tip 508 may also selectively extend distally relative to the outer band 506 to cut and separate tissue from the lead. The cutting tip 508 and the inner sheath also define an inner lumen 510 for receiving such a lead.

The distal end portion 500 of the lead extraction device further includes a first imaging device 512, a second imaging device 514, a third imaging device 516, and a fourth imaging device 518, which may specifically be any of the imaging devices described herein. Generally, the imaging devices 512, 514, 516, and 518 send signals corresponding to an image of the treatment space, and a display in operative communication with the imaging devices 512, 514, 516, and 518 (shown elsewhere) provides the image of the treatment space to a user. The imaging devices 512, 514, 516, and 518 are carried by the outer band 506. The first imaging device 512 and the second imaging device 514 are disposed in and provide the image of the treatment space in a first viewing plane 520. The third imaging device 516 and the fourth imaging device 518 are disposed in and provide the image of the treatment space in a second viewing plane 520 that is substantially perpendicular to the first viewing plane 520 (that is, perpendicular ±5 degrees). In some embodiments, the imaging devices 512, 514, 516, and 518 may be recessed into the outer band 506 to inhibit abrasion of the imaging devices 512, 514, 516, and 518 during advancement of the vascular treatment device within a subject. In some embodiments, the distal end portion 500 includes only the first imaging device 512 and the second imaging device 514. The imaging devices 512, 514, 516, and 518 may advantageously require relatively low amounts of power for image acquisition and generation, and the imaging devices 512, 514, 516, and 518 may advantageously require relatively few operative connections to other components, thereby simplifying manufacturing. The imaging devices 512, 514, 516, and 518 may facilitate providing relatively simple images that are easy for a user to understand and interpret.

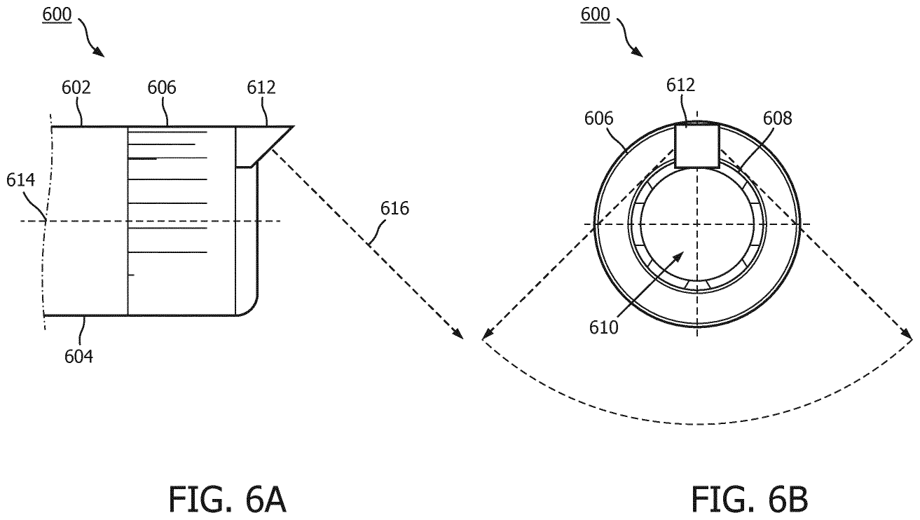

As another example and referring to FIGS. 6A and 6B, an exemplary embodiment of a distal end portion 600 of a lead extraction device is illustrated. The distal end portion 600 is part of a sheath assembly 602 that includes an outer sheath 604 or jacket and an outer band or distal tip 606 coupled to and extending distally from the outer sheath 604. An inner sheath (not shown) is rotatably carried within the outer sheath 604, and a cutting tip 608 couples to and extends distally from the inner sheath. As such, the cutting tip 608 is rotatable relative to the outer band 606 to cut and separate tissue from an adjacent lead. The cutting tip 608 may also selectively extend distally relative to the outer band 606 to cut and separate tissue from the lead. The cutting tip 608 and the inner sheath also define an inner lumen 610 for receiving such a lead.

The distal end portion 600 of the lead extraction device further includes an imaging device 612, which may specifically be any of the imaging devices described herein. Generally, the imaging device 612 send a signal corresponding to an image of the treatment space, and a display in operative communication with the imaging device 612 (shown elsewhere) provides the image of the treatment space to a user. The imaging device 612 has an atraumatic shape that extends distally relative to the outer band 606 and is disposed radially aside of a longitudinal axis 614 of the sheath assembly 602. In some embodiments, the imaging device 612 is partially recessed in the outer band 606. The imaging device 612 may be disposed to provide the image of the treatment space with an acute viewing centerline 616 relative to the longitudinal axis 614 of the sheath assembly 602. The imaging device 612 may provide a viewing cone of ±45 degrees from the centerline 616.

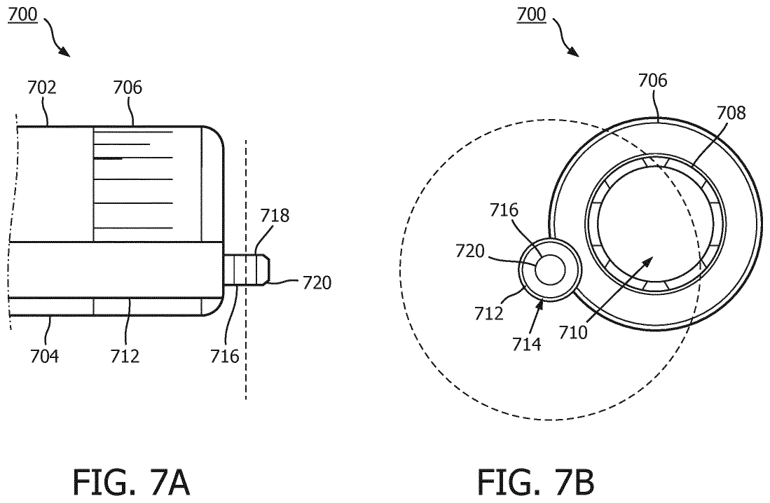

As another example and referring to FIGS. 7A and 7B, an exemplary embodiment of a distal end portion 700 of a lead extraction device is illustrated. The distal end portion 700 is part of a sheath assembly 702 that includes an outer sheath 704 or jacket and an outer band or distal tip 706 coupled to and extending distally from the outer sheath 704. An inner sheath (not shown) is rotatably carried within the outer sheath 704, and a cutting tip 708 couples to and extends distally from the inner sheath. As such, the cutting tip 708 is rotatable relative to the outer band 706 to cut and separate tissue from an adjacent lead. The cutting tip 708 may also selectively extend distally relative to the outer band 706 to cut and separate tissue from the lead. The cutting tip 708 and the inner sheath also define an inner lumen 710 for receiving such a lead.

The sheath assembly 702 of the lead extraction device further includes an auxiliary sheath 712 coupled to the outer sheath 704 and the outer band 706. The auxiliary sheath 712 may be disposed outwardly from the outer sheath 704 and the outer band 706, as illustrated, or inwardly of the outer sheath 704 and the outer band 706. The auxiliary sheath 712 includes an auxiliary lumen 714 that translatably carries an imaging catheter 716. The imaging catheter 716 carries an imaging device 718 at a distal end portion 720. The imaging device 718 may specifically be any of the imaging devices described herein. Generally, the imaging device 718 send a signal corresponding to an image of the treatment space, and a display in operative communication with the imaging device 718 (shown elsewhere) provides the image of the treatment space to a user. The imaging device 718 may be a distally-viewing imaging device, a transversely-viewing imaging device, or both a distally-viewing and transversely-viewing imaging device. In some embodiments, the imaging catheter 716 may include one or more markers and/or fluoroscopy may be used to facilitate registering the imaging device 718 relative to the cutting tip 708. In some embodiments, a mechanical registering mechanism (not shown) may be used to register an imaging plane to the cutting tip 708. In some embodiments, the imaging catheter 716 may be selectively fixable relative to the auxiliary sheath 712.

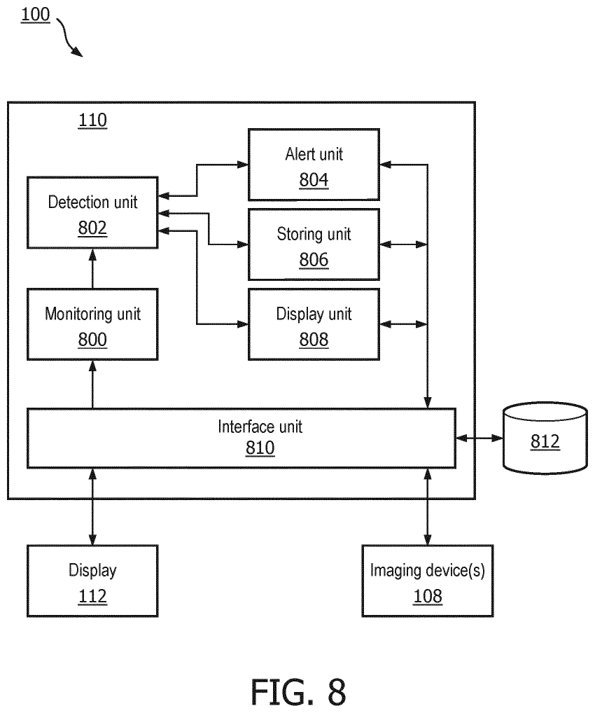

FIG. 8 illustrates an exemplary configuration of the controller 110 of the vascular treatment system 100 shown in FIG. 1. In one embodiment, the controller 110 can be a portion of a vascular device configured to identify at least one feature sensed by the vascular device. For example, the controller 110 allows the user to view one or more images of the treatment space during a medical operation, such as a lead extraction procedure. In FIG. 8, the controller 110 includes a monitoring unit 800, a detection unit 802, an alert unit 804, a storing unit 806, a display unit 808, and an interface unit 810.

As used herein, the term “unit” may refer to, be part of, or include an Application Specific Integrated Circuit (ASIC), an electronic circuit, a processor or microprocessor (shared, dedicated, or group) and/or memory (shared, dedicated, or group) that executes one or more software or firmware programs, a combinational logic circuit, and/or other suitable components that provide the described functionality. Thus, while this disclosure includes particular examples and arrangements of the units, the scope of the present system should not be so limited since other modifications will become apparent to the skilled practitioner.

Although these sub-units 800-810 are illustrated as children units subordinate of the parent unit (e.g., controller 110), each sub-unit can be operated as a separate unit from the controller 110, and other suitable combinations of sub-units are contemplated to suit different applications. For example, one or more units can be selectively bundled as a key software model running on the processor having software as a service (SaaS) feature.

All relevant information can be stored in a central database 812, e.g., as a non-transitory data storage device and/or a machine-readable data storage medium carrying computer-readable information and/or computer-executable instructions, for retrieval by the controller 110 and its children units. The interface unit 810 is configured to provide an interface between the controller 110, the central database 812, and other relevant devices or systems related to the vascular treatment system 100, such as the display 112 and the imaging devices 108.

The interface unit 810 controls operation of, for example, the display 112, and other related system devices, services, and applications. The other devices, services, and applications may include, but are not limited to, one or more software or hardware components, etc., related to the controller 110. The interface unit 810 also receives data, signals, or parameters from the vascular treatment system 100, such as the imaging devices 108, which are communicated to the respective units, such as the controller 110, and its children units 800-810.

The monitoring unit 800 is configured to receive the data, signals, and parameters from the imaging devices 108 via the interface unit 810, and to provide imaging information during medical operations, such as lead extraction procedures. Specifically, the monitoring unit 800 provides detailed imaging information using at least one signal received from at least one imaging device 108. In one embodiment, the imaging device 108 is configured to be disposed in a vascular space of a patient and to send at least one signal corresponding to an image of the vascular space.

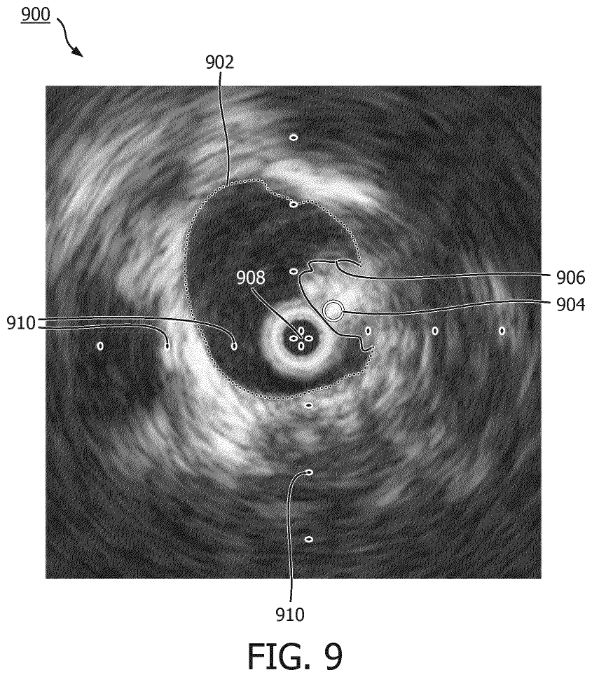

The detection unit 802 is configured to examine the data, signals, and parameters received from the monitoring unit 800, such as an image signal, for detecting any anatomical features, such as all vascular anatomical features, lead segments, and one or more anomalies. For example, an anomaly can be caused by unwanted movement of the lead or surrounding materials, such as calcium or thrombus accumulations near the lead. During operation, the detection unit 802 performs a feature recognition technique related to each vascular space and identifies one or more features of the corresponding vascular space based on a predetermined analysis. Specifically, the controller 110 having the detection unit 802 is electronically coupled to the imaging device 108, and is configured to receive at least one signal corresponding to the image of the vascular space. The detection unit 802 is configured to determine at least one feature included in the image of the vascular space and identify at least a portion of the feature using a graphical representation, such as a visible mark and/or the like. Detailed descriptions of the feature recognition technique are provided below in paragraphs related to FIGS. 9-14.

In one embodiment, the detection unit 802 is configured to recognize the anomaly of the corresponding vascular space using a machine learning analysis. For example, the machine learning analysis can be a supervised learning process used in a data mining method based on sample input and output values. Training data having a set of exemplary categories can be used to generate an inferred function of determining a probable configuration of the vascular space. The detection unit 802 can learn from the training data based on predetermined categories to recognize the configuration of the vascular space. For example, based on a color or shape of the features associated with the training data, the detection unit 802 can recognize the configuration of the lead and the calcium accumulations near the lead.

In another example, the machine learning analysis can include a fuzzy set, a codified weighted grading system, a ranking method, and the like. Each identified anomaly can be recorded and stored in the central database 812. In one example, the central database 812 is a relational database storing data associated with the features detected in the vascular space. In some embodiments, each feature is ranked with a weighted score to quantify a degree of the anomaly caused by the unwanted movement or surrounding materials.

For example, the detection unit 802 is configured to generate the weighted score of the identified anomaly using decision tree logic. For example, the decision tree logic includes control charts, Chi-square Automatic Interaction Detector, Iterative Dichotomiser 3, Multivariate Adaptive regression Spines, and the like. Other suitable machine learning technologies are also contemplated to suit different applications. In embodiments, the detection unit 802 is configured to determine a likelihood of anomaly based on the weighted score.

The alert unit 804 is configured to inform the user or other related systems of the detected anomaly. One or more messages can be sent by the alert unit 804 to a mobile device or any computing device, such as the display 112, to alert the user or other related systems. For example, when the anomaly is detected, the alert unit 804 can generate a visual, textual, haptic and/or audible signal, indicator, or notification to inform the user of the detected anomaly.

The storing unit 806 is configured to control and digitally store relevant information related to the controller 110 in the central database 812. More specifically, the central database 812 includes any information related to the vascular space having analysis data about anomaly incidents, users, medical events, other data, signals, and parameters associated with the vascular space, etc. Further, other relevant medical data can be stored in the central database 812 for the purposes of research, development, improvement of the comparative logic or algorithms and further investigations. For example, for the machine learning process, the storing unit 806 can store historical data related to tracking of location/shape changes of a vessel wall boundary 902, a lead 904 (e.g., a cardiac lead), and an adhesion 906 (e.g., FIG. 9) over a predetermined period. The lead 904 can be a cardiac (e.g., pacemaker) lead but in various embodiments, the lead 904 can be a lead of any medical device to suit different applications.

The display unit 808 is configured to interactively display an appropriate status or information message and/or image associated with the anomaly for illustration on the display 112. In embodiments, the display unit 808 is configured to instruct the display 112 to output the graphical representation identifying the portion of the feature in the vascular space to the display 112. In one embodiment, a screen shot related to one or more anomalies is displayed on the display 112 for viewing. For example, the display unit 808 can instruct the display 112 to display an intra- and/or extra-vascular anatomy associated with the vascular space, along with leads, various anatomical features, such as adhesions (e.g., thrombus, vegetations, calcium, etc.), and other extraction tools. In another embodiment, a report related to each anomaly is generated by the display unit 808, and also automatically transmitted to a medical agency or other entities, as desired.

FIGS. 9-13C illustrate exemplary images of a vascular space 900 generated by the monitoring unit 800 using the imaging device 108. In embodiments, the imaging device 108 can use any suitable imaging techniques, such as a visible light, an ultrasound, an optical coherence tomography, an impedance mapping, and the like. For example, during the lead extraction procedure, an ultrasound array associated with the imaging device 108 can provide front and/or lateral views in the vascular space 900. Each image of the vascular space 900 is displayed on the display 112 for viewing by the user.

FIG. 9 illustrates the vascular space 900 showing a vessel wall boundary 902, a lead 904, and an adhesion 906. In one embodiment, the detection unit 802 determines at least one of: the vessel wall boundary 902, the lead 904, and the adhesion 906, such as fibrotic adhesions to cardiovascular segments, calcium in fibrotic adhesions, thrombus within cardiovascular segments, vegetation within cardiovascular segments, and boundaries between a vessel wall and at least one of pericardium and pleura.

In some embodiments, the imaging device 108 is an ultrasound device, and further includes the acoustic lens 418 coupled to the ultrasound device, as shown in FIG. 4A. Other suitable arrangements shown in FIG. 3-7 are also contemplated to suit different applications. In embodiments, the detection unit 802 determines at least one feature included in the image of the vascular space 900 and identifies at least a portion of the feature using a graphical representation (e.g., the vessel wall boundary 902, the lead 904, and the adhesion 906). In the illustrated embodiments of FIGS. 9-13C, the detection unit 802 identifies the portion of the feature with an identifiable line.

Returning to FIG. 9, in one embodiment, the identifiable line is a colored line. For example, the vessel wall boundary 902 can be depicted with a RED dotted line, the lead 904 can be depicted with a YELLOW solid line, and the adhesion 906 can be depicted with a GREEN solid line. Other suitable colors and line types, such as blue dashed lines, can be used to depict other features in the vascular space 900 to suit the application. For example, other anatomical features in cardiovascular features of interest in the vascular space 900 can be displayed using different identifiable lines. These lines can be displayed statically, dynamically, pulsing, or of varying transparencies and brightness to suit the application.

In some embodiments, different shades of colors can be used to display the anatomical features in the vascular space 900. For example, the virtual histology using the different shades of WHITE color scheme can be used to correlate the shades with different tissue types based on a reflectance of an ultraviolet light. As such, boarders, tissue types, and geometric configurations of the anatomical features in the vascular space 900 can be distinctively displayed on the display 112.

In another example, the feature is at least partially surrounded by the identifiable line. For example, an outer periphery following a profile of an inner vasculature wall, such as the vessel wall boundary 902, can be at least partially surrounded by the RED dotted line. In another example, a cross-sectional shape of the lead 904 can be at least partially surrounded by the YELLOW solid line. In yet another example, a peripheral edge of the adhesion 906 can be at least partially surrounded by the GREEN solid line. In one embodiment, these lines can be displayed in various line weights as differentiators.

In FIG. 9, the feature is also at least partially overlaid by the identifiable line. For example, an outer periphery following a profile of an inner vasculature wall, such as the vessel wall boundary 902, can be at least partially overlaid by the RED dotted line. In another example, a cross-sectional shape of the lead 904 can be at least partially overlaid by the YELLOW solid line. In yet another example, a peripheral edge of the adhesion 906 can be at least partially overlaid by the GREEN solid line.

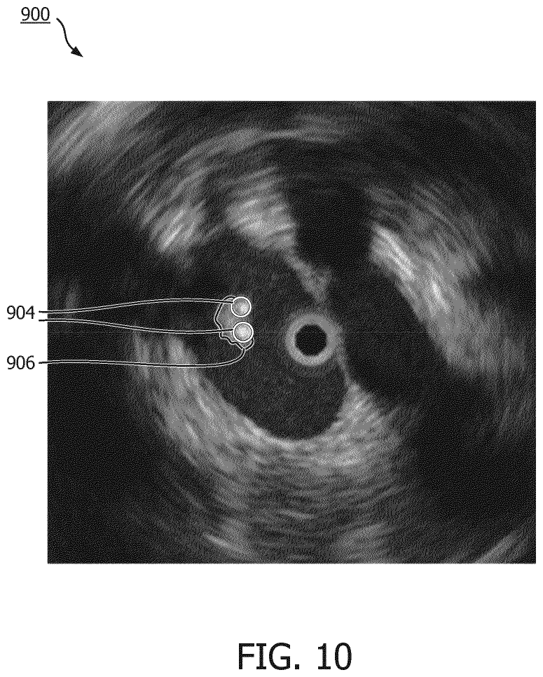

As shown in FIG. 9, the image of the vascular space 900 provides vascular lumen boundaries, vessel wall boundaries, and locations of leads being extracted during the lead extraction procedure. Thus, using the signal corresponding to the image of the vascular space 900, the detection unit 802 provides the detailed imaging information regarding a presence of fibrotic adhesions to the leads 904, a presence of fibrotic adhesions 906 to cardiovascular segments, a presence of thrombus or vegetation within the cardiovascular segments, a presence of calcium in fibrotic adhesions to either tissue or leads, and boundaries between a vessel wall and pericardium/pleura. For example, FIG. 9 shows the lead 904 is embedded at least partially inside the adhesion 906, and FIG. 10 shows a lead-on-lead adhesion formation where two leads 904 are embedded at least partially inside the adhesion 906.

Returning to FIG. 9, in various embodiments, the detection unit 802 is configured to perform a calculation of a distance between the vessel wall boundary 902 and the lead 904, and a calculation of a pericardial space and/or a vessel wall thickness (e.g., for monitoring for effusion). To perform the calculations, another graphical representation, such as a center marker 908 having intersecting vertical and horizontal hairlines, is used to identify a center point of the image of the vascular space 900. One or more dots 910 are overlaid on at least a portion of the image of the vascular space 900. The detection unit 802 is configured to generate distance scale information that represents an actual size of a distance between two reference locations in a numerical value (e.g., 0.5 millimeter).

For example, the two reference locations can be the vessel wall boundary 902 and the lead 904. In another example, the two reference locations can be the center marker 908 and one of the dots 910. In yet another example, the two reference locations can be any two of the dots 910. Although the dots 910 are shown separately and independently, the dots 910 can be a part of any points in the image of the vascular space 900, such as the vessel wall boundary 902, the lead 904, and the adhesion 906

In still another example, the two reference locations can be the vessel wall boundary 902 and the adhesion 906. As such, any two identifiable points in the image of the vascular space 900 can be used as reference locations. The detection unit 802 is configured to calculate the actual size based on a number of pixels disposed between the two reference locations in the image of the vascular space 900.

For example, when the two reference locations 902, 904 are identified, the detection unit 802 can generate the distance scale information representative of the distance between the two reference locations 902, 904 based on the number of pixels. In one example, the user or other system may enter the actual size of the distance in the numerical value relative to the number of pixels (e.g., 1 millimeter per 1000 pixels). The detection unit 802 is configured to record data related to the distance scale information in a lookup table stored in the central database 812. To determine the feature included in the image of the vascular space 900, the detection unit 802 can subsequently access the lookup table to calculate the distance. As another example, the lookup table can include information about different types of anatomical features identified in the image of the vascular space 900.

To determine the actual size, the detection unit 802 can count a number of pixels disposed between the two reference locations 902, 904. The detection unit 802 can proportionally extrapolate a pixel ratio relative to the entered size to calculate the distance of any two reference locations by performing a linear transformation. Using this pixel ratio extrapolation technique, the detection unit 802 can generate the distance scale information.

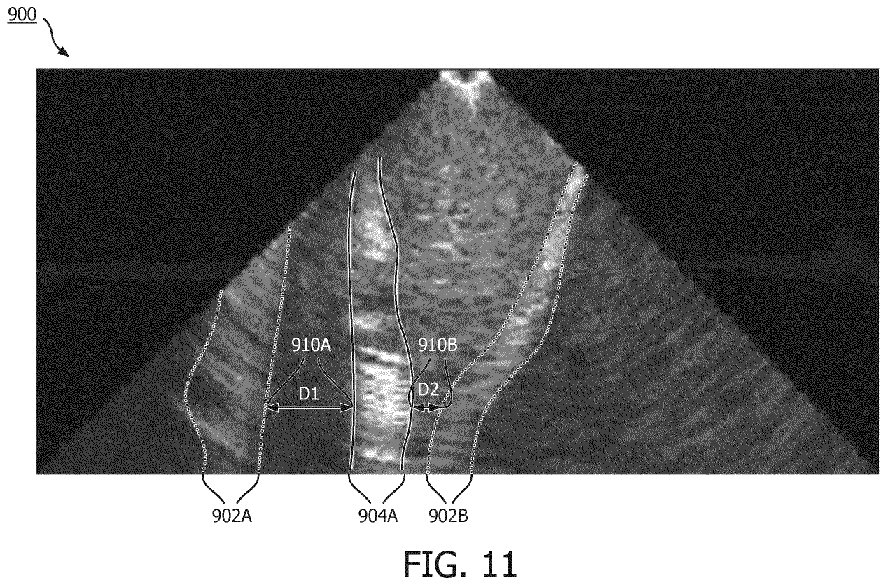

FIG. 11 illustrates another exemplary image of the vascular space 900 generated by the monitoring unit 800 using the imaging device 108 featuring two vessel wall boundaries 902A, 902B and a lead 904A. In the vascular space 900 of FIG. 11, a first vessel wall boundary 902A is shown on a left side of the lead 904A and a second vessel wall boundary 902B is shown on a right side of the lead 904A. The detection unit 802 overlays the image with a plurality of dots 910A, 910B on at least a portion of the image of the vascular space 900, such as the vessel wall boundaries 902A, 902B and the lead 904A, to determine at least two reference locations.

In embodiments, the detection unit 802 identifies the first two reference locations 910A between the first vessel wall boundary 902A and the lead 904A to calculate a first distance D1 between two dots 910A. In another example, the detection unit 802 identifies the second two reference locations 910B between the second vessel wall boundary 902B and the lead 904A to calculate a second distance D2 between two dots 910B. The detection unit 802 calculates the first and/or second distance D1, D2 between two dots 910A, 910B using the pixel ration extrapolation technique.

For example, the detection unit 802 determines the distance D1 between two dots 910A and calculates the first distance D1 between the first vessel wall boundary 902A and the lead 904A using the determined distance between the two dots 910A based on the number of pixels disposed between the two dots 910A. Similarly, the detection unit 802 determines the distance D2 between two dots 910B and calculates the second distance D2 between the second vessel wall boundary 902B and the lead 904A using the determined distance between the two dots 910B based on the number of pixels disposed between the two dots 910B.

Using the same technique, the detection unit 802 can also calculate one or more dimensions of at least one feature shown in the image of the vascular space 900 using the determined distance between any two dots. In various embodiments, the dots 910A, 910B can be a part of any features in the image of the vascular space 900, such as the vessel wall boundary 902, the lead 904, the adhesion 906, and the like.

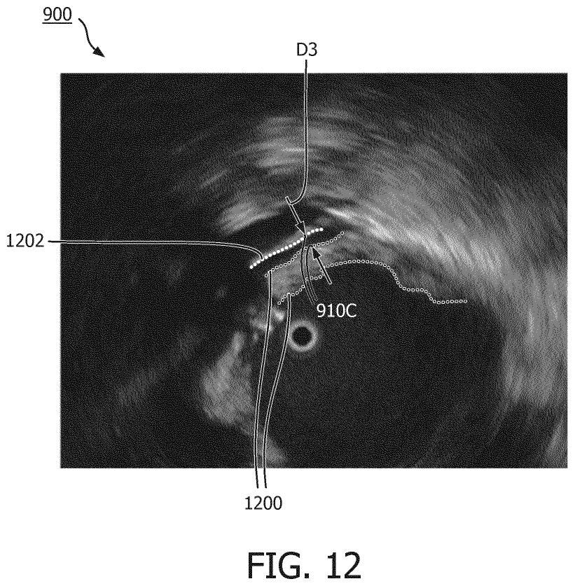

FIG. 12 illustrates yet another exemplary image of the vascular space 900 generated by the monitoring unit 800 using the imaging device 108 featuring an atrial wall 1200 and a pericardium 1202. In FIG. 12, the detection unit 802 identifies third two reference locations 910C between the atrial wall 1200 and the pericardium 1202 to calculate a third distance D3 between two dots 910C. The detection unit 802 determines the third distance D3 between the two dots 910C and calculates the distance D3 between the atrial wall 1200 and the pericardium 1202 using the determined distance between the two dots 910C based on the number of pixels disposed between the two dots 910C. For example, the calculated third distance D3 can be used to monitor an occurrence of pericardial effusion.

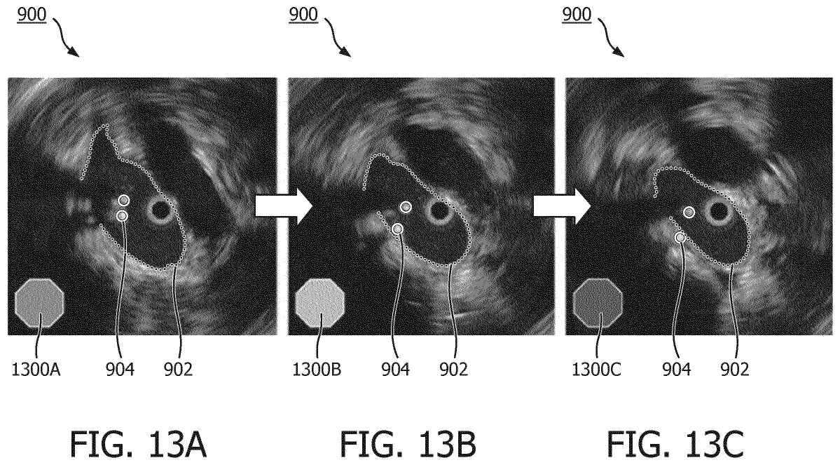

FIGS. 13A-13C illustrate exemplary notifications generated by the alert unit 804 to inform the user of the detected anomaly in the vascular space 900. In FIG. 13, one or more indicators, such as a GREEN indicator 1300A, a YELLOW indicator 1300B, and a RED indicator 1300C, can be used to output the notification. The alert unit 804 is configured to output the notification when at least one event occurs in the image of the vascular space 900 using the GREEN, YELLOW, and/or RED indicators 1300A, 1300B, 1300C. In some embodiments, these indicators 1300A, 1300B, 1300C can have various shapes and sizes to suit different applications. In various embodiments, the alert unit 804 is configured to determine locations of any features shown in the image of the vascular space 900, such as the lead 904 and the vessel wall boundary 902.

In FIG. 13A, the alert unit 804 instructs the display 112 to display the GREEN indicator 1300A to inform the user of a first event occurrence. For example, the first event refers to no anomaly in the image of the vascular space 900 because the leads 904 are safely disposed entirely within the vessel wall boundary 902. In FIG. 13B, the alert unit 804 instructs the display 112 to display the YELLOW indicator 1300B to inform the user of a second event occurrence. For example, the second event refers to a potential or prospective anomaly in the image of the vascular space 900 because one of the leads 904 is disposed close to the vessel wall boundary 902. In FIG. 13C, the alert unit 804 instructs the display 112 to display the RED indicator 1300C to warn the user of a third event occurrence. For example, the third event refers to an unwanted anomaly in the image of the vascular space 900 because one of the leads 904 may extend outside of the vessel wall boundary 902.

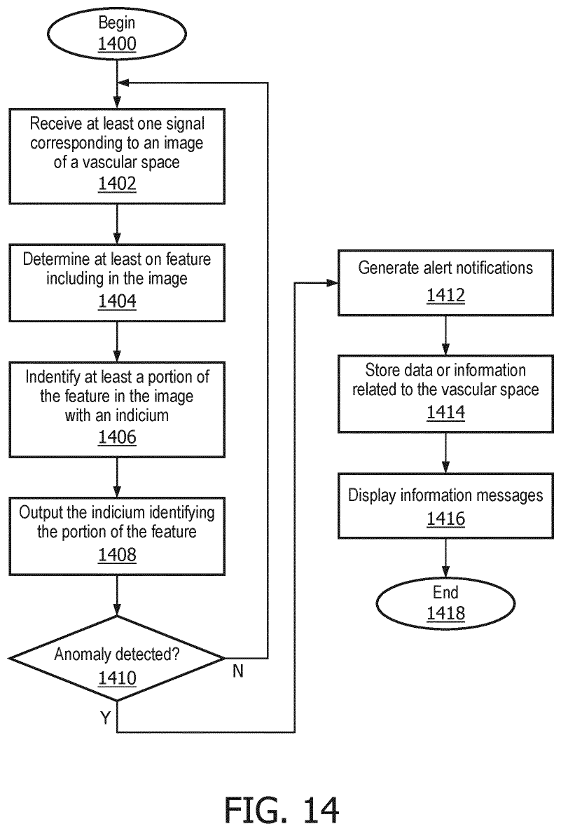

FIG. 14 illustrates an exemplary method or process of identifying anatomical features sensed by the controller 110 of the vascular treatment system 100 shown in FIG. 8. Although the following steps are primarily described with respect to the embodiments of FIGS. 1-13C, it should be understood that the steps within the method may be modified and executed in a different order or sequence without altering the principles of the present disclosure.

The method begins at step 1400. In step 1402, the monitoring unit 800 receives at least one signal corresponding to an image sensed by a vascular device, such as the vascular treatment system 100. For example, the monitoring unit 800 provides detailed imaging information using the at least one signal received from the imaging device 108 configured to be disposed in the vascular space 900.

In step 1404, the detection unit 802 determines at least one feature included in the image of the vascular space 900 based on the signal received from the monitoring unit 800. For example, the detection unit 802 performs a feature recognition technique related to the vascular space 900 and identifies anatomical features in the image of the vascular space 900 based on the feature recognition technique.

In step 1406, the detection unit 802 identifies at least a portion of the feature in the image of the vascular space 900 with a graphical representation. For example, the detection unit 802 determines at least one feature included in the image of the vascular space 900 and identifies at least a portion of the feature using a colored line (e.g., the vessel wall boundary 902, the lead 904, and the adhesion 906).

In step 1408, the display unit 808 outputs the graphical representation identifying the portion of the feature to a display device based on the feature identified by the detection unit 802. For example, data relating to one or more features identified by the detection unit 802 can be transmitted to the display unit 808, and the display unit 808 instructs the display 112 to output the data relating to the graphical representation on the display 112 via the interface unit 810. In another example, data relating to one or more features identified by the detection unit 802 can be transmitted to the storing unit 806. The storing unit 806 stores the data in the central database 812 via the interface unit 810. The display unit 808 retrieves the data from the central database 812 via the interface unit 810, and instructs the display 112 to output the data relating to the graphical representation on the display 112 via the interface unit 810.

In step 1410, the detection unit 802 examines the features in the image of the vascular space 900 and detects an anomaly based on the identified features. For example, the detection unit 802 performs a feature recognition technique related to each vascular space and identifies the anomaly of the corresponding vascular space based on a predetermined analysis. When the anomaly is detected, control proceeds to step 1412. Otherwise, control returns to step 1402.

In step 1412, the alert unit 804 generates one or more notifications regarding the features in the image of the vascular space 900. For example, the alert unit 804 generates one or more messages or signals, such as the GREEN, YELLOW, and/or RED indicators 1300A, 1300B, 1300C by comparing relative locations of the lead 904 and the vessel wall boundary 902.

In step 1414, the storing unit 806 stores relevant data or information related to the image of the vascular space 900 along with analysis data about all events, such as the anomaly incidents, users, medical events, other data, signals, and parameters associated with the vascular space, etc. in the central database 812 for subsequent retrieval or processing.

In step 1416, the display unit 808 interactively displays an appropriate status or information message and/or image associated with the vascular space 900 for illustration. For example, the display unit 808 can display a report about the events occurred in the vascular space 900 on the display 112. In another example, the report can be printed or transmitted to another system for additional processing. The method ends at step 1418 or returns to step 1402.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A vascular device configured to identify features sensed by the vascular device, comprising:
an imaging device configured to be disposed in a vascular space and send at least one signal corresponding to an image of the vascular space; and
a processing device electronically coupled to the imaging device, the processing device configured to:
receive the at least one signal corresponding to the image of the vascular space;
determine at least one feature included in the image;
identify at least a portion of the feature using a graphical representation; and
output the graphical representation identifying the portion of the feature to a display device.

2. The vascular device of claim 1, wherein the imaging device is an ultrasound device, and further comprising an imaging device coupled to the ultrasound device.

3. The vascular device of claim 1, wherein to identify the portion of the feature with the graphical representation, the processing device identifies the portion of the feature with an identifiable line.

4. The vascular device of claim 3, wherein the identifiable line is a colored line.

5. The vascular device of claim 3, wherein the feature is at least partially surrounded by the identifiable line.

6. The vascular device of claim 3, wherein the feature is at least partially overlaid by the identifiable line.

7. The vascular device of claim 1, wherein to determine the at least one feature, the processing device determines at least one of: a vessel wall boundary, a lead, fibrotic adhesions to cardiovascular segments, calcium in fibrotic adhesions, thrombus within cardiovascular segments, vegetation within cardiovascular segments, and boundaries between a vessel wall and at least one of pericardium and pleura.

8. The vascular device of claim 1, wherein the processing device is further configured to:
overlay the image with a plurality of dots;
determine at least one distance between two dots of the plurality of dots; and
calculate dimensions of the at least one feature using the determined distance between the two dots.

9. The vascular device of claim 1, wherein the processing device is further configured to:
overlay the image with a plurality of dots;
determine at least one distance between two dots of the plurality of dots; and
calculate a distance between two features of the at least one feature using the determined distance between the two dots.

10. The vascular device of claim 1, wherein to determine the at least one feature included in the image, the processing device uses machine learning.

11. The vascular device of claim 1, wherein to determine the at least one feature included in the image, the processing device access a lookup table.

12. The vascular device of claim 1, wherein the processing device is further configured to output a notification when at least one event occurs.

13. The vascular device of claim 12, wherein to determine the at least one feature, the processing device determines a lead and a vessel wall, and one of the at least one event occurs when the lead extends outside the vessel wall.

14. A method for identifying features sensed by the vascular device, the method comprising:
receiving at least one signal corresponding to an image sensed by a vascular device;
determining at least one feature included in the image;
identifying at least a portion of the feature with a graphical representation; and
outputting the graphical representation identifying the portion of the feature to a display device.

15. The method of claim 14, wherein identifying a portion of the feature with the graphical representation comprises identifying the portion of the feature with an identifiable line.

16. The method of claim 15, wherein identifying the portion of the feature with an identifiable line comprises surrounding the feature with the identifiable line.

17. The method of claim 15, wherein identifying the portion of the feature with an identifiable line comprises overlaying the feature with the identifiable line.

18. The method of claim 14, further comprising:
overlaying the image with a plurality of dots;
determining at least one distance between two dots of the plurality of dots; and
calculating dimensions of the at least one feature using the determined distance between the two dots.

19. The method of claim 14, further comprising:
overlaying the image with a plurality of dots;
determining at least one distance between two dots of the plurality of dots; and
calculating a distance between two features included in the image using the determined distance between the two dots.

20. The method of claim 14, further comprising outputting a notification when at least one event occurs.
